SEQUENCE LISTING (6 sequences)

SEQ ID NO: 1 - DNA, 1321 bp, Arabidopsis thalania
  Intron (148)..(311)
atggcaccggtgactgaagtttctccgatgatcaacacattggaagtcgcagatgacaaaatgacgaatctcagttcctctgggaaacctccaagaaacatctctgcaatgcgccattgcaacagcactgcctggttgaccgattccgttggttcatctcttctttgcttttttttttttttaaattcaaatgcttgtgattgtaattgcacgatcacgaacagataagataatgtagcttanatcggatctcgtgactcaaaaacaatccaaagtttgaaacttacaatgtgaaaatgatttgtttgcaggaaggagatgagagatttggtgcaaaatctccggaaggagtaaattcaacattccagcctgtgtttagatctggaagttggtctgataaaggaccaaaacaatcaatggaagatgaattcatttgtgttgatgatctcactgagtatattggatcatccactggagctttctatggggtatataagatttgcttcatcgaatatttcaaaaacgttctcttttttaaaatgattgtttgtttatgaaggtgtttgatggacacggaggtgttgatgctgcatcattcacaaaaaagaacattatgaagcttgtaatggaagacaaacatttcccaaccagcacaaagaaggcaacaaggagtgcttttgtgaagactgatcatgctttggctgatgcttcttctcttgatagatcctctggcaccactgcactcactgctctcattttggacaagactatgttgattgcaaacgctggtgactccagagcggtgcttgggaaacgaggcagagccattgagttatcaaaggaccacaaacctaactgtacttccgagcggctacgtattgagaagcttggaggtgtgatctacgatggttaccttaacgggcagctatctgtggctcgagccttaggagattggcacattaagggaactaaaggttcactctgcccgctcagctgcgagcctgagctggaagagattgtgctgacggaagaggatgagtatcttataatgggctgtgatggactctgggatgtgatgagtagccagtgtgcagtgacgatggtgaggagggagctgatgcagcataatgatcctgagagatgctctcaagctttggttaaggaggctcttcaacgaaacagctgtgacaatcttactgtagtggttgtctgtttctcgcccgaagcgcctcctcggattgagattcccaagtcgcataagaggaggagcatctctgcagagggattggatctcctcaaaggtgttttgaacgagttgtga

SEQ ID NO: 2 - protein, 361 aa, Arabidopsis thalania
MAPVTEVSPMINTLEVADDKMTNLSSSGKPPRNISAMRHCNSTAWLTDSEGDERFGAKSPEGVNSTFQPVFRSGSWSDKGPKQSMEDEFICVDDLTEYIGSSTGAFYGVFDGHGGVDAASFTKKNIMKLVMEDKHFPTSTKKATRSAFVKTDHALADASSLDRSSGTTALTALILDKTMLIANAGDSRAVLGKRGRAIELSKDHKPNCTSERLRIEKLGGVIYDGYLNGQLSVARALGDWHIKGTKGSLCPLSCEPELEEIVLTEEDEYLIMGCDGLWDVMSSQCAVTMVRRELMQHNDPERCSQALVKEALQRNSCDNLTVVVVCFSPEAPPRIEIPKSHKRRSISAEGLDLLKGVLNEL

SEQ ID NO: 3 - DNA, 1313 bp, Arabidopsis thalania
  CDS (54)..(1136)
aaaaaagagatgactctgacaaatttaagaaagcttttgatcggaatctggaaatggcaccggtgactgaagtttctccgatgatcaacacattggaagtcgcagatgacaaaatgacgaatctcagttcctctgggaaacctccaagaaacatctctgcaatgcgccattgcaacagcactgcctggttgaccgattccgaaggagatgagagatttggtgcaaaatctccggaaggagtaaattcaacattccagcctgtgtttagatctggaagttggtctgataaaggaccaaaacaatcaatggaagatgaattcatttgtgttgatgatctcactgagtatattggatcatccactggagctttctatggggtgtttgatggacacggaggtgttgatgctgcatcattcacaaaaaagaacattatgaagcttgtaatggaagacaaacatttcccaaccagcacaaagaaggcaacaaggagtgcttttgtgaagactgatcatgctttggctgatgcttcttctcttgatagatcctctggcaccactgcactcactgctctcattttggacaagactatgttgattgcaaacgctggtgactccagagcggtgcttgggaaacgaggcagagccattgagttatcaaaggaccacaaacctaactgtacttccgagcggctacgtattgagaagcttggaggtgtgatctacgatggttaccttaacgggcagctatctgtggctcgagccttaggagattggcacattaagggaactaaaggttcactctgcccgctcagctgcgagcctgagctggaagagattgtgctgacggaagaggatgagtatcttataatgggctgtgatggactctgggatgtgatgagtagccagtgtgcagtgacgatggtgaggagggagctgatgcagcataatgatcctgagagatgctctcaagctttggttaaggaggctcttcaacgaaacagctgtgacaatcttactgtagtggttgtctgtttctcgcccgaagcgcctcctcggattgagattcccaagtcgcataagaggaggagcatctctgcagagggattggatctcctcaaaggtgttttgaacgagttgtgaaggaactgagagcaagttggaaaaaggtaagaagagttactttattctttttttgtttcctcttttgataggaaagtcagagagatattcaaggttgtgtgcttttttctctggctttccttccaagttttgattgattccattttttgtaaaaaaaaaaaaaaaaaaaaaaaa

SEQ ID NO: 4 - protein, 361 aa, Arabidopsis thalania; identical to SEQ ID NO: 2

SEQ ID NO: 5 - DNA, 1086 bp, Arabidopsis thalania
  CDS (1)..(1083)
atggcaccggtgactgaagtttctccgatgatcaacacattggaagtcgcagatgacaaaatgacgaatctcagttcctctgggaaacctccaagaaacatctctgcaatgcgccattgcaacagcactgcctggttgaccgattccgaaggagatgagagatttggtgcaaaatctccggaaggagtaaattcaacattccagcctgtgtttagatctggaagttggtctgataaaggaccaaaacaatcaatggaagatgaattcatttgtgttgatgatctcactgagtatattggatcatccactggagctttctatggggtgtttgatggacacggaggtgttgatgctgcatcattcacaaaaaagaacattatgaagcttgtaatggaagacaaacatttcccaaccagcacaaagaaggcaacaaggagtgcttttgtgaagactgatcatgctttggctgatgcttcttctcttgatagatcctctggcaccactgcactcactgctctcattttggacaagactatgttgattgcaaacgctggtgactccagagcggtgcttgggaaacgaggcagagccattgagttatcaaaggaccacaaacctaactgtacttccgagcggctacgtattgagaagcttggaggtgtgatctacgatggttaccttaacgggcagctatctgtggctcgagccttaggagattggcacattaagggaactaaaggttcactctgcccgctcagctgcgagcctgagctggaagagattgtgctgacggaagaggatgagtatcttataatgggctgtgatggactctgggatgtgatgagtagccagtgtgcagtgacgatggtgaggagggagctgatgcagcataatgatcctgagagatgctctcaagctttggttaaggaggctcttcaacgaaacagctgtgacaatcttactgtagtggttgtctgtttctcgcccgaagcgcctcctcggattgagattcccaagtcgcataagaggaggagcatctctgcagagggattggatctcctcaaaggtgttttgaacgagttgtga

SEQ ID NO: 6 - protein, 361 aa, Arabidopsis thalania; identical to SEQ ID NO: 2

(19) United States
(12) Patent Application Publication
(10) Pub. No.: US 2003/0014776 A1
(43) Pub. Date: Jan. 16, 2003

(54) Maternal effect gametophyte regulatory polynucleotide

(76) Inventors: Ueli Grossniklaus, Uster (CH); Norbert Huck, Zurich (CH); James M. Moore, Zumikon (CH)

Correspondence Address: MCKEE, VOORHEES & SEASE, P.L.C., 801 GRAND AVENUE, SUITE 3200, DES MOINES, IA, 50309-2721, US

(21) Appl. No.: 10/178,977

(22) Filed: Jun. 25, 2002

Related U.S. Application Data
(60) Provisional application No. 60/300,624, filed on Jun. 25, 2001.

(51) Int. Cl. A01H 5/00; C12P 21/02; C07H 21/04; C12N 9/24; C12N 5/04

(52) U.S. Cl. 800/287; 435/69.1; 435/200; 435/419; 435/320.1; 536/23.2

(57) ABSTRACT

This invention relates to the isolation and characterization of a novel gene from Arabidopsis with maternal gametophyte control of pollen tube development and sperm release. The novel gene and gene product may be used to manipulate the function of gametophytes, fertilization and pollination for the generation of apomixis in Arabidopsis and other plant types. Novel sequences, polypeptides and methods of use for the same are disclosed.

### CROSS-REFERENCE TO RELATED APPLICATIONS

This application claims benefit under 35 U.S.C. §119(e) of provisional application 60/300,624 filed Jun. 25, 2001.

### FIELD OF THE INVENTION

This invention relates to the isolation and characterization of a novel gene from Arabidopsis with maternal gametophytic control of pollen tube development and sperm release. The novel gene and gene product may be used to manipulate the function of gametophytes, pollination, fertilization and seed development for the generation of apomixis in Arabidopsis and other plant types. Mutations of this gene lead to normal pollen tube guidance to the micropyle but no double fertilization occurs.

### BACKGROUND OF THE INVENTION

The plant life cycle alternates between a haploid and diploid generation, the gametophyte producing the gametes and the sporophyte generating the spores. The entire pollination/fertilization process includes pollination, pollen germination and growth, pollen tube guidance to the embryo sac, pollen tube disruption usually occurring in one of the synergids (Russell, S. D. 1992), sperm cell release, targeting to the egg and central cell, and finally the fusion of the two pairs of gametes. In the last years, the interest has mainly focused on self-incompatibility systems (reviewed in Dickinson 2001, Brugiere et al. 2000) and pollen tube guidance (reviewed in Lord 2000, Franklin-Tong 1999). To date, little is known about the complex mechanisms that target the pollen tube to the embryo sac, the interactions between female and the male gametophytes, sperm discharge, the targeting of the sperm cells to egg and central cell, respectively, and gametic and nuclear fusion.

In flowering plants, double fertilization initiates seed development. While one sperm fertilizes the egg cell to form the zygote, a second sperm fuses with the bi-nucleate central cell to give rise to the triploid endosperm. Embryo and endosperm coordinately develop embedded in maternal tissues of sporophytic origin that will eventually form the seed coat. The molecular mechanisms activating reproductive development are largely unknown and the interactions between the various seed tissues remain a complex and unresolved problem.

The double fertilization process results in a diploid embryo and a triploid endosperm tissue, which are otherwise genetically identical. Endosperm has 2n of the maternal genome and 1 n of the paternal genome. It has been shown that such a unique genetic composition is important to normal seed development (Rhoades and Dempsey, Genetics 54:505-22 (1966); Lin, Genetics 107:103-15 (1984); Scott et al., Development 125:3329-41 (1998)).

The term apomixis is generally accepted as the replacement of sexual reproduction by various forms of asexual reproduction (Rieger et al., In Glossary of Genetics and Cytogenetics, Springer-Verlag, New York, N.Y., 1976). In general, the initiation of cell proliferation in the embryo and endosperm are uncoupled from fertilization. In most forms of apomixis, however, pseudogamy or fertilization of the polar nuclei to produce endosperm is necessary for seed viability. Apomixis has great economic potential because it can cause any genotype, regardless of how heterozygous, to breed true. It is a reproductive process that bypasses female meiosis and syngamy to produce embryos genetically identical to the maternal parent. With apomictic reproduction, progeny of specially adaptive or hybrid genotypes would maintain their genetic fidelity throughout repeated life cycles. In addition to fixing hybrid vigor, apomixis can make possible commercial hybrid production in crops where efficient male sterility or fertility restoration systems for producing hybrids are not known or developed. Apomixis can make hybrid development and breeding more efficient. It also simplifies hybrid production and increases genetic diversity in plant species with good male sterility.

Introducing the apomictic trait into normally sexual crops has been attempted. Asker (Heredias, Vol. 91, 231-241, 1979) reports that attempts have been unsuccessful with wheat, sugar beets, and maize. PCT publication WO 89/00810 (Maxon et al, 1989) discloses inducing an apomictic form of reproduction in cultivated plants using extracts from nondomesticated sterile alfalfa plants. When induction of male sterility was evaluated in sorghum, sunflower, pearl millet, and tomato it was reported that there was reduced seed set in sorghum, pearl millet, and sunflower and reduced fruit set in tomato.

Although apomixis is effectively used in Citrus to produce uniform and disease-and virus-free rootstock (Parlevliet JE et al, in Citrus. Proc. Am. Soc. Hort. Sci., Vol. 74, 252-260, 1959) as well as in buffelgrass (Bashaw, Crop Science, Vol. 20, 112, 1980) and Poa (Pepin et al, Crop Science, Vol. 11, 445-448, 1971) to produce improved cultivars, it has not been successfully transferred to other cultivated crop plants.

Imprinting also presents hurdles for the engineering of apomixis. Recently, it has become evident that maternal effects, which can be of gametophytic or sporophytic origin, play an important role in seed development. Imprinting is crucial for normal endosperm development in cereals. In maize, a strict dependence for a 2m:1p ratio of maternal to paternal genomes in maize endosperm has been shown and any deviation thereof leads to seed abortion. Such an imprinting barrier is of relevance to the engineering of apomixis technology. While autonomous development of both embryo and endosperm exists in some apomictic species, such autonomous development is relatively rare, especially among the grasses. Instead, most apomicts require fertilization of the endosperm (pseudogamy) for successful seed formation which poses a problem for the transfer of apomixis to sexual crops: The fertilization of an unreduced central cell with a normal reduced sperm will yield a 4m:1p ratio of maternal to paternal genomes which is expected to result in seed abortion. This may be the primary reason for the high degree of seed sterility observed in apomictic hybrids obtained in introgression programs.

To overcome this imprinting barrier, natural apomicts have evolved two strategies to prevent seed abortion due to unbalanced endosperm genome dosage: 1) Seed development is insensitive to altered ratios of maternal to paternal genomes, or 2) gametogenesis or double fertilization has been modified to yield genetically balanced endosperm. Successful engineering of apomixis in sexual crop plants, such as the cereals, will require understanding of the molecular genetics of genomic imprinting in seed development.

The generation of apomixis is only one of the many potential benefits of manipulating the double fertilization process. Induction of parthenocarpy for seedless fruits and vegetables or the production of value added custom seeds which involve enhancement of certain tissue areas at the expense of others is another.

As can be seen there is a continuing need in the art to identify and understand genes and their products which control the fundamental aspects of seed development including 1) the activating signals at fertilization, 2) the control of cell proliferation in embryo and endosperm, 3) the interaction between the different tissues of the seed, and 4) the role of genomic imprinting for seed development in sexual and apomictic species.

It is thus an object of the present invention to provide a novel gene and protein which regulates the interaction between male and female gametophytes and fertilization.

It is yet another object of the invention to provide a DNA sequence which encodes a gene from Arabidopsis which is involved with gametophyte development.

A further object of the present invention is to provide constructs for expression of or inhibition of this gene product.

A further object is to provide models, compositions and methods for generating apomixis in plants and/or for further understanding the roles of various products in the fertilization process in plants.

Finally, it is an object of the present invention to provide genetic material which can be used to screen other genomes to identify other genes with similar effects from other plant sources or even from animal sources.

### SUMMARY OF THE INVENTION

According to the invention a novel gene involved in maternal control of gametophyte development has been isolated and characterized from Arabidopsis. The gene encodes a protein product which is intimately involved in the regulation of the pollination/fertilization process. Mutants with disruptions in the gene demonstrated aberrant pollen tube development leading to prevention of fertilization.

Thus, the novel gene and protein product of the invention provide a valuable tool for the manipulation of maternal gametophyte development to induce parthenocarpy, apomixis, plant sterility or even to engineer the specific content of valuable components of seeds. Genetic engineering methods known in the art can be used to inhibit expression of the gene or to further induce expression thus controlling the developmental effects regulated thereby, in not only Arabidopsis but other plants and animals. Further, due to the highly conserved nature of this family of genes, it is expected that other such genes may be identified using the DNA and amino acid sequences herein to characterize other closely related genes from other species with similar effects.

### DETAILED DESCRIPTION OF THE FIGURES

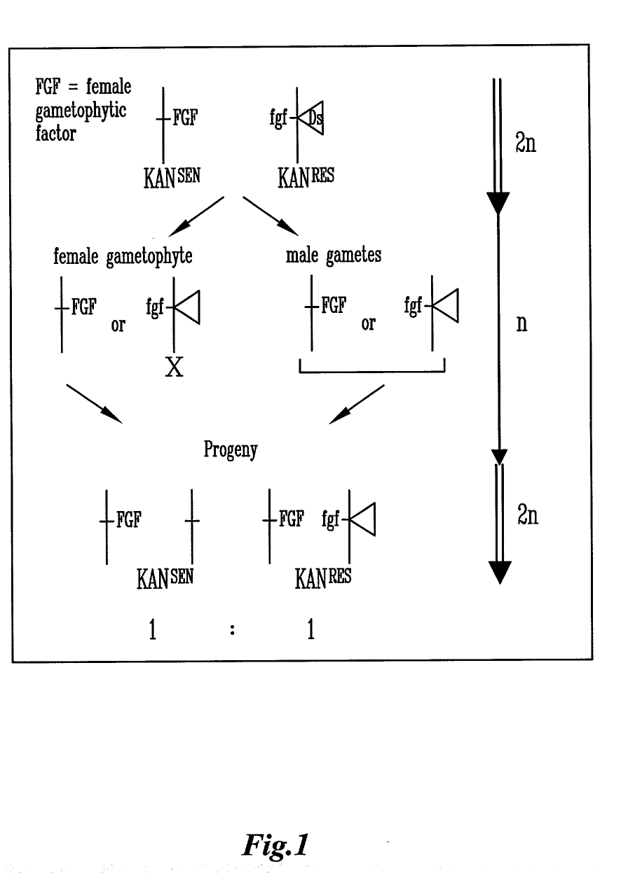

FIG. 1 is a schematic showing screening strategy for female gametophytic mutants based on segregation ratio distortion.

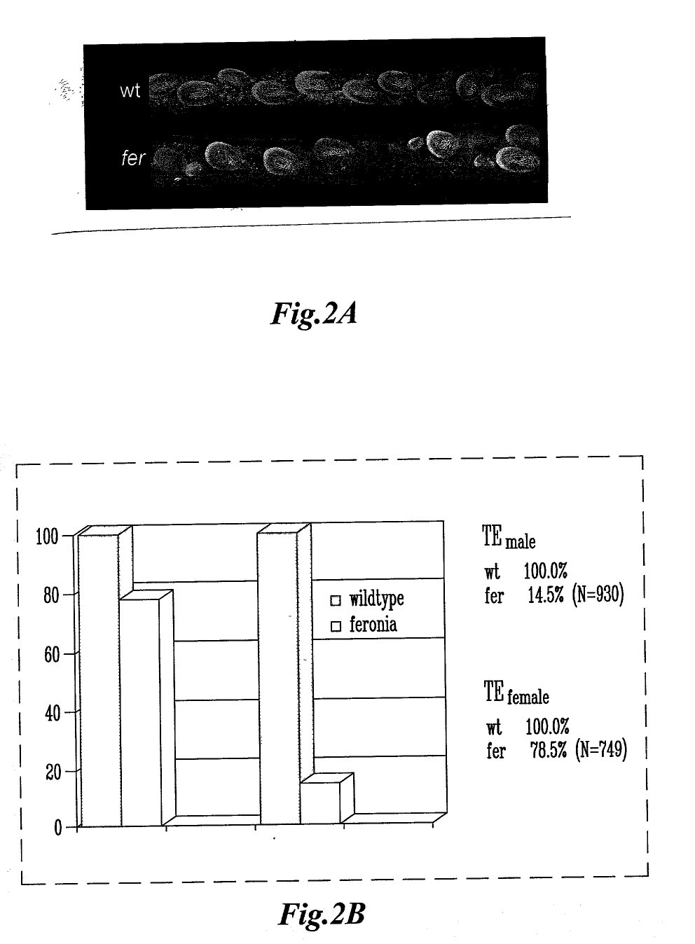

FIGS. 2(a-b) show that feronia is a semisterile mutant and shows a drastically reduced transmission efficiency through the female gametophyte. (a) Opened siliques of feronia and wild type about 4 days after pollination. In feronia (fer) half of the ovules are unfertilized. (b) Transmission efficiency (TE) of feronia compared to the wild type (wt).

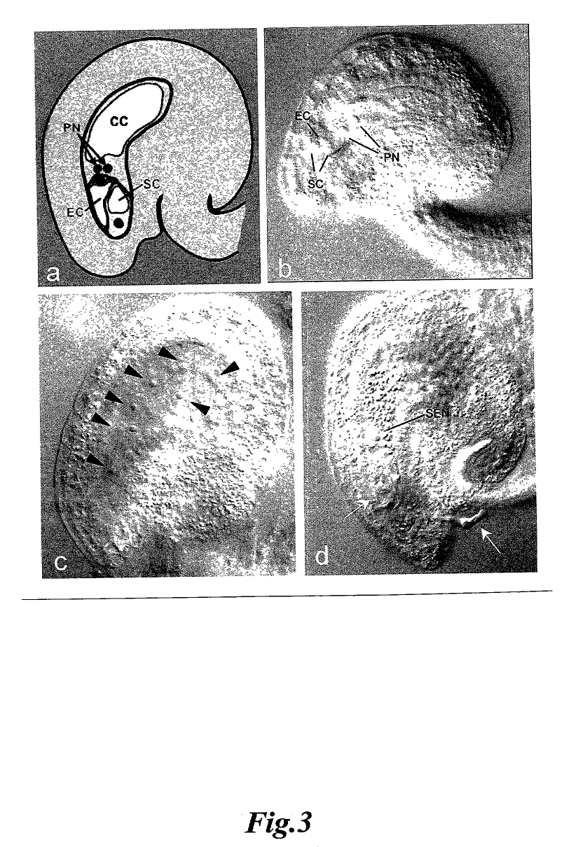

FIGS. 3(a-d) show whole mount preparations of wild type and mutant ovules. (a) Diagram of normally developed embryo sac before fertilization (modified from Drews 1998). (b) Whole mount preparation of an ovule of the feronia mutant before fertilization. At this stage all ovules of the feronia mutant show no morphological differences to ovules of wild-type plants. (c) Wild-type ovule around 24 h after fertilization. Free nuclear endosperm (arrowheads) is already present. (d) Aberrant ovule in the feronia mutant. The secondary endosperm nucleus has not divided, even the pollen tube (arrow) has reached the ovule and entered the embryo sac. Inferonia mutants about one half of the ovule show this phenotype.

Abbreviations: CC, central cell; E, egg cell; PN polar nuclei; PT, pollen tube; SC, synergid cell, SEN, secondary endosperm nucleus.

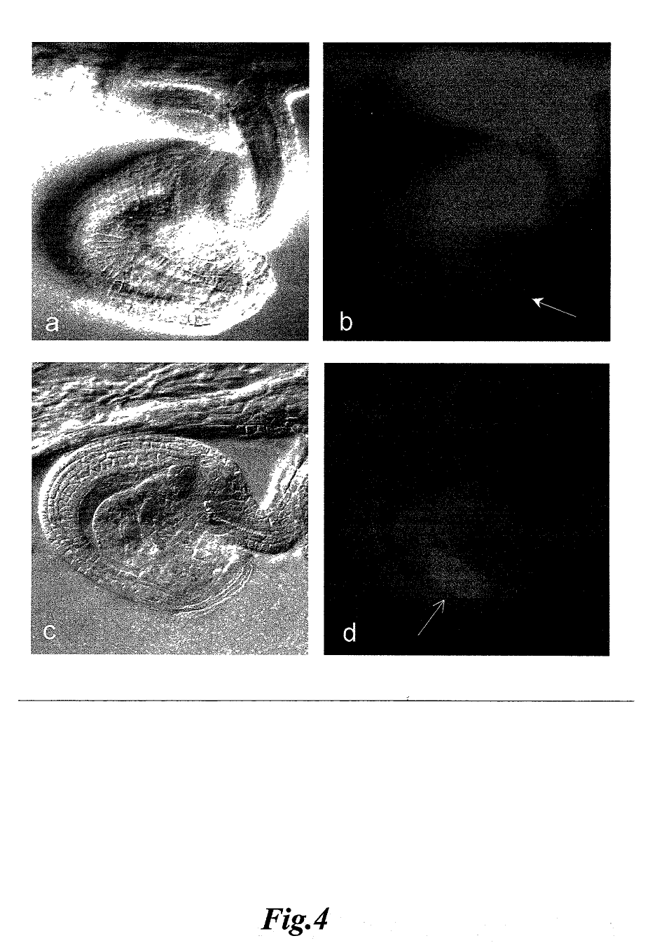

FIGS. 4(a-d) show whole mount preparations stained with aniline blue. The pollen tube shows a bluish-green fluorescence. (a,b) Wild-type ovule. (a) DIC image. (b) Fluorescence image. The pollen tube enters the micropyle and terminates in the synergid (arrow). (c,d) Mutant ovule. (c) DIC image. (d) Fluorescence image. The pollen tube enters the micropyle and winds around the egg apparatus (arrow) without discharging the sperm cells.

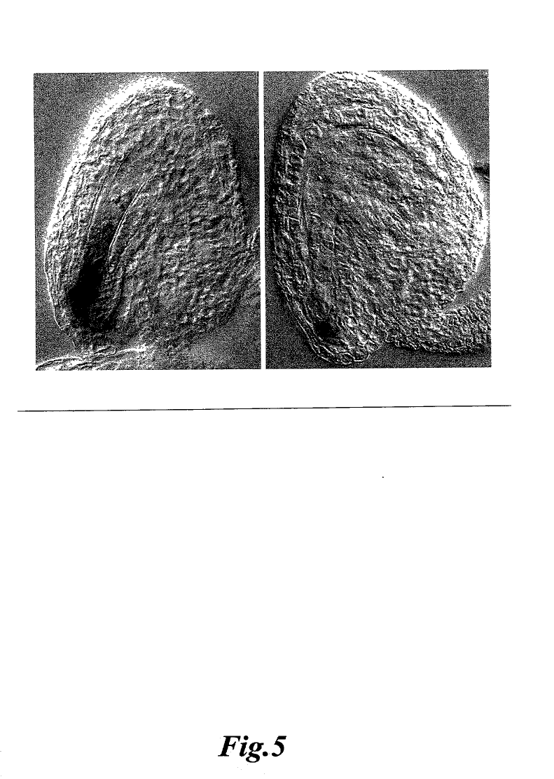

FIGS. 5(a-b) show crosses of feronia to a pollen marker line. The marker line is wild-type at the feronia locus. (a) Mutant ovule. The pollen enters the embryo sac and winds around the egg apparatus. Because the pollen carries the wild-type allele, the feronia phenotype determined by the female gametophyte. (b) Wild-type ovule. The pollen tube terminates in the synergid.

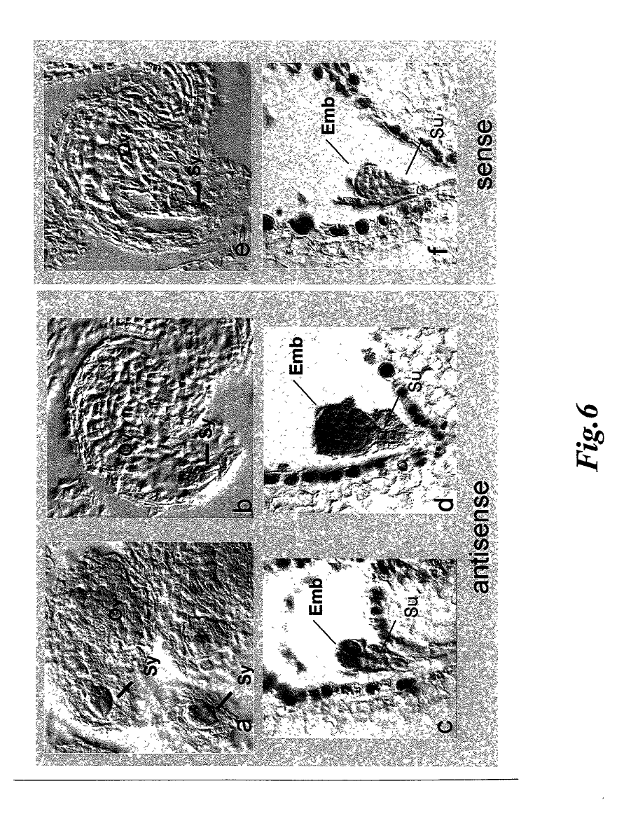

FIGS. 6(a-d) show expression of FERONIA as detected by in situ hybridization. (a-d) Tissue probed with labeled antisense RNA. (a,b) Mature ovule before fertilization. The signal is very strong in the synergids of the female gametophyte. (c) Early globular embryo with strong expression in the embryo proper. (d) Mid-globular embryo showing expression in embryo proper and suspensor. (e-f) Tissue probed with labeled sense RNA. (e) Mature embryo around fertilization. (f) Early globular embryo.

Abbreviations: Ec, egg cell; Emb, embryo proper; Ov, ovule; Su, suspensor; Sy, synergid

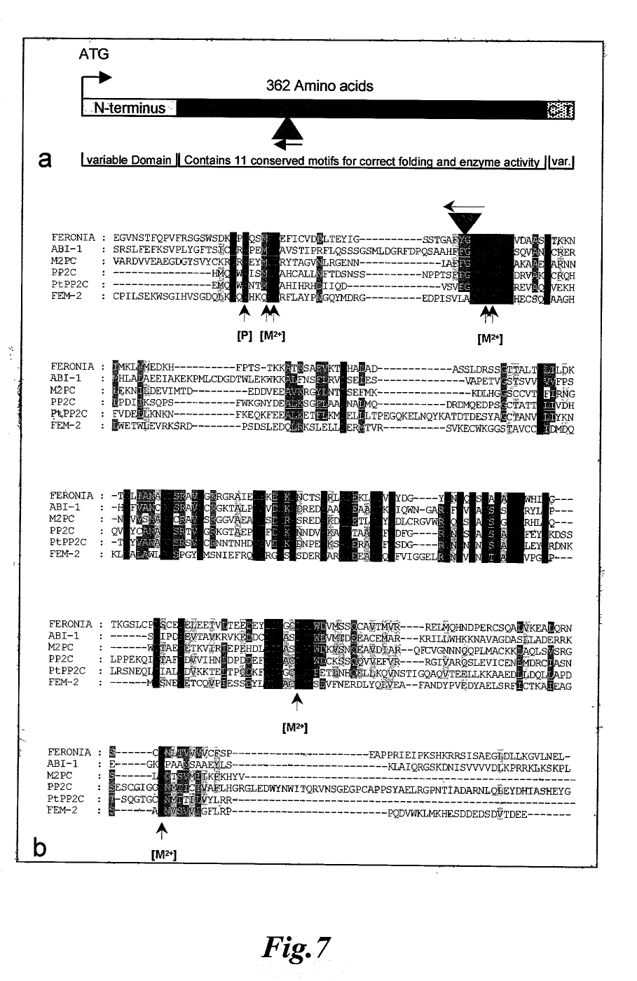

FIGS. 7(a-b) show that the Ds-element in the feronia mutant disrupts a Protein Phosphatase 2C. (a) Schematic drawing of the Protein Phosphatase 2C gene harboring the Ds-element. (b) Alignment of the PP2C core with other characterized PP2Cs: ABI-1: Abscisic acid insensitive-1 from Arabidopsis thaliana, Accession-No.P49597 (Meyer et al. 1994); MP2C: Protein Phosphatase 2C from Medicago sativa, Accession-No. Y11607 (Meskiene et al. 1998); PP2C: PP2C-2 from Schizosaccharomyces pombe, Accession-No. Q09172 (Shiozaki and Russell 1995); PtPP2C: Protein Phosphatase 2C form Paramecium tetrauelia, Accession-No. P49444 (Klumpp, et al. 1994), FEM2: FEM-2 from Caenorhabditis elegans, Accession-No. P49594 (Pilgrim et al. 1995). The green arrows indicate the conserved metal binding sites, the red arrow the site interacting with the phosphate-group (Das et al. 1996). The Ds-element inserted close to a conserved metal coordination site.

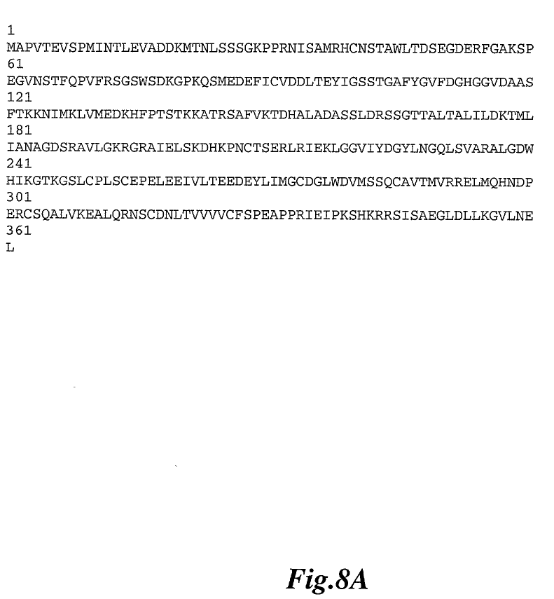

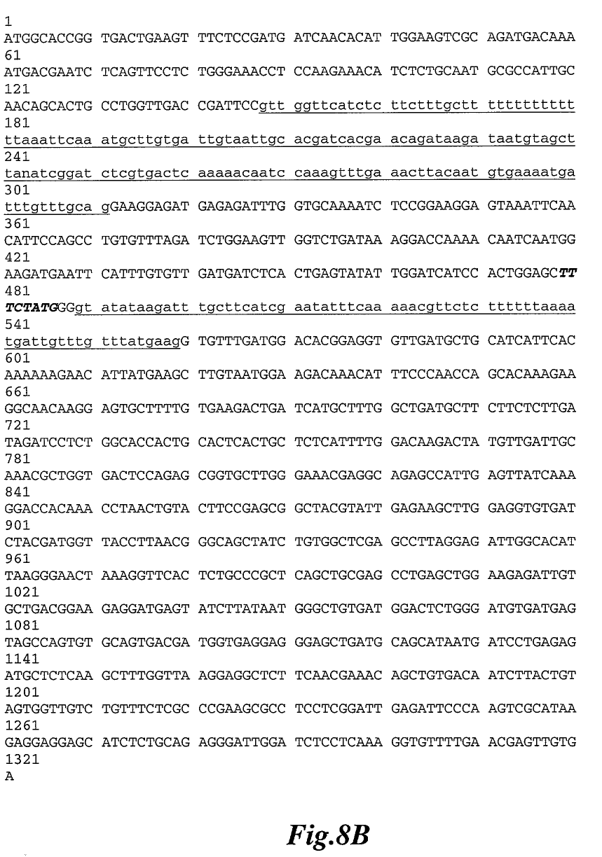

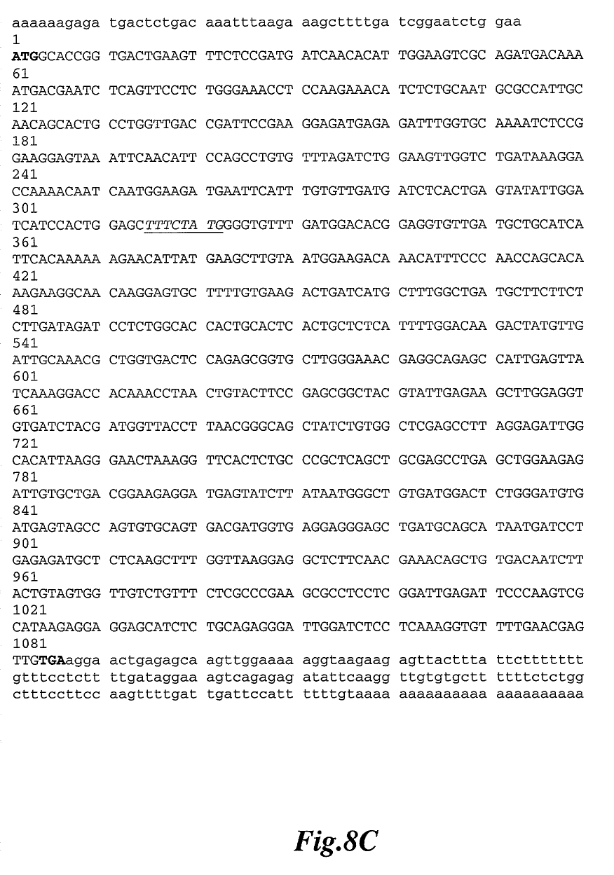

FIG. 8(a-c) show the sequence information of the FERONIA gene. (a) Translation of the cDNA sequence into a protein sequence. The asterisk marks the position of the Ds element in the feronia mutant. (b) Genomic sequence of the FERONIA Phosphatase 2C gene of sequence a. The introns are underlined and written in small caps. The target site of the Ds element is italicized and in bold. (c) cDNA sequence of the FERONIA protein phosphatase gene disrupted by the Ds element in the feronia mutant. The 5′ and 3′ UTR are written in small letters. The start codon and the stop codon are printed in bold letters. The underlined eight bases represent the target site of the Ds element.

### DETAILED DESCRIPTION OF THE INVENTION

Applicants have discovered a regulatory gene (termed FERONIA) isolated from Arabidopsis that is involved in maternal control of gametophyte development. The FERONIA gene product is a protein phosphatase and functions as a component of a signaling pathway between the female and male gametophyte involved in pollen tube rupture and sperm release If the gene product is missing, the pollen tube does not provide sperm cells for fertilization.

Thus, in one embodiment of the invention, the gene or its product can be used to control pollen tube development to tailor plants to specific requirements and in one embodiment provide for clonal propagation of seeds. The gene or its product can be used in regulation of sperm release to direct release of sperm to specific cell types in the female gametophyte. This regulation makes the manipulation of double fertilization possible to generate apomixis, as the production of viable apomictic seeds usually requires the formation of sexual endosperm.

This invention further contemplates methods of controlling expression of these regulatory genes in plants through genetic engineering techniques which are known and commonly used by those of skill in the art. Such methods include but are in no way limited to generation of apomixis, generation of a parthenocarpic phenotype, control of undesirable seeds, generation of seeds engineered to produce higher endosperm content and concomitant higher byproduct content such as proteins or lipids, as well as other tissue specific regulation based upon expression of the gene at time, spatial and developmental periods.

The FERONIA gene product is expressed in the embryo sac of mature ovules and in developing seeds during the reproductive phase of development. In the embryo sac very strong expression was detected in the synergids. Thus the invention also contemplates temporal and spatial promoter regions and regulatory elements natively associated with the FERONIA gene which are capable of providing tissue and developmentally specific expression of operably linked sequences to seed development, fertilization, female gametophyte development and the like.

The present invention provides polynucleotides, related polypeptides and all conservatively modified variants of a newly discovered FERONIA sequences from Arabidopsis. According to the invention, a novel protein phosphatase FERONIA gene has been identified which regulates the male and female gametophyte interaction in Arabidopsis. The full length nucleotide sequence of the FERONIA gene comprises the sequence found in SEQ ID NO: 1, the cDNA is SEQ ID NO: 2 and the cDNA with coding only nucleotides is SEQ ID NO: 5.

Therefore, in one aspect, the present invention relates to an isolated nucleic acid comprising an isolated polynucleotide sequence encoding a FERONIA protein. In a further aspect, the present invention is selected from: (a) an isolated polynucleotide encoding a polypeptide of the present invention; (b) a polynucleotide having at least 70% identity to a polynucleotide of the present invention; (c) a polynucleotide comprising at least 25 nucleotides in length which hybridizes under high stringency conditions to a polynucleotide of the present invention; (d) a polynucleotide comprising a polynucleotide of the present invention; and (e) a polynucleotide which is complementary to the polynucleotide of (a) to (e).

In another aspect, the present invention relates to a recombinant expression cassette comprising a nucleic acid as described. Additionally, the present invention relates to a vector containing the recombinant expression cassette. Further, the vector containing the recombinant expression cassette can facilitate the transcription and translation of the nucleic acid in a host cell. The present invention also relates to the host cells able to express the polynucleotide of the present invention. A number of host cells could be used, such as but not limited to, microbial, mammalian, plant, or insect.

In yet another embodiment, the present invention is directed to a transgenic plant or plant cells, containing the nucleic acids of the present invention. Preferred plants containing the polynucleotides of the present invention include but are not limited to Arabidopsis, maize, soybean, sunflower, sorghum, canola, wheat, alfalfa, cotton, rice, barley, tomato, and millet. In another embodiment, the transgenic plant is a maize plant or plant cells. In yet another embodiment are the seeds from the transgenic plant.

This invention also provides an isolated polypeptide comprising (a) a polypeptide comprising at least 70% sequence identity to a polypeptide of the present invention; and (b) a polypeptide encoded by a nucleic acid of the present invention.

Another embodiment of the subject invention is a host cell stably transformed by a polynucleotide construct as described above, and a method of making a polypeptide of a recombinant gene comprising:

a) providing a population of these host cells; and

b) growing the population of cells under conditions whereby the polypeptide encoded by the coding sequence of the expression cassette is expressed;

c) isolating the resulting polypeptide.

A number of expression systems using the said host cells could be used, such as but not limited to, microbial, mammalian, plant, or insect.

Also included in yet another embodiment are regulatory regions capable of conferring spatial and temporal expression that are fertilization or gametophyte development specific. These comprise regulatory elements such as promoters that are natively associated with the nucleotide sequences encoding the proteins of the invention as well as their functional equivalents. In addition to these promoter sequences, the promoters of the invention encompass fragments and variants of these particular promoters as defined herein. Further the nucleotide sequences encoding the proteins disclosed herein can be used to isolate promoters of the genes of the invention using standard molecular protocols as described and incorporated by reference herein. These promoter elements can also be used to isolate other signaling components associated with regulation of fertilization, and can be used to engineer synthetic fertilization-regulatory promoters.

In yet another object of the invention, the feronia gene can be used and manipulated to generate apomixis in plants. According to the invention, the FERONIA gene product induces the release of sperm cells to the synergid. If one could inhibit the function or FERONIA gene product, as demonstrated herein, the sperm cells are not released into the syngergid. Further in order to generate apomixis, one could use a central cell-specific or inducible promoter to cause expression of FERONIA to promote sperm release by the pollen tube into the central cell rather than the synergid. This would provide for normal endosperm with a 4m:2p maternal to paternal genome ratio in an apomictically engineered plant, which is necessary for the generation of normal seed set. Any of a number of other transgenic techniques may be utilized to generate sterile plants as disclosed herein with the loss of function manipulation, or as models to discover stimulants of inhibitors of maternal control of pollen tube development or sperm release in plants.

### DEFINITIONS

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

As used herein, “antisense orientation” includes reference to a duplex polynucleotide sequence that is operably linked to a promoter in an orientation where the antisense strand is transcribed. The antisense strand is sufficiently complementary to an endogenous transcription product such that translation of the endogenous transcription product is often inhibited.

As used herein, “chromosomal region” includes reference to a length of a chromosome that may be measured by reference to the linear segment of DNA that it comprises. The chromosomal region can be defined by reference to two unique DNA sequences, i.e., markers.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (B);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W. H. Freeman and Company.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). Thus, the maize preferred codon for a particular amino acid may be derived from known gene sequences from maize. Maize codon usage for 28 genes from maize plants are listed in Table 4 of Murray et al., supra.

As used herein “full-length sequence” in reference to a specified polynucleotide or its encoded protein means having the entire amino acid sequence of, a native (non-synthetic), endogenous, biologically active form of the specified protein. Methods to determine whether a sequence is full-length are well known in the art including such exemplary techniques as northern or western blots, primer extensions, S1 protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous” in reference to a nucleic acid, is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT U.S. patent application Ser. No. 93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “localized within the chromosomal region defined by and including” with respect to particular markers includes reference to a contiguous length of a chromosome delimited by and including the stated markers.

As used herein, “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes of that pair to be followed. A genotype may be defined by use of one or a plurality of markers.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2^{nd} ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference t a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” can include reference to whole plants, plant parts or organs (e.g., leaves, stems, roots, etc.), plant cells, seeds and progeny of same. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants. Particularly preferred plants include maize, soybean, sunflower, sorghum, canola, wheat, alfalfa, cotton, rice, barley, and millet.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNa that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether nor not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, preferably 90% sequence identity, and most preferably 100% sequence identity (i.e., complementary) with each other.

The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1X to 2X SSC (20X SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5X to 1X SSC at 55 to 50° C. Exemplary high stringency conditions include hybridization in 50% formamide, 1 M NaCl, 1% SDS at 37° C., and a wash in 0.1X SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (%GC)−0.61 (% form) −500/L; where M is the molarity of monovalent cations, %GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acids Probes, Part I, Chapter 2, Ausubel, et al., Eds., Greene Publishing and Wiley-Interscience, New York (1995).

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York(1995).

Unless otherwise stated, sequence identity/similarity values provided herein refer to the value obtained using the BLAST 2.0 suite of programs using default parameters. Altschul et a., Nucleic Acids Res. 25:3389-3402 (1997). Software for performing BLAST analyses is publicly available, e.g., through the National Center for Biotechnology-Information (http://www.hcbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(I) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, ore preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

(e)(ii) The terms “substantial Identity” in the context of a peptide indicates that a peptide comprises a sequence with at least 70% sequence identity to a reference sequence, preferably 80%, ore preferably 85%, most preferably at least 90% or 95% sequence identity to the reference sequence over a specified comparison window. Optionally, optimal alignment is conducted using the homology alignment algorithm of Needleman and Wunsch, J. Mol. Biol. 48:443 (1970). an indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions which are not identical may differ by conservative amino acid changes.

The invention in one aspect comprises expression constructs comprising a DNA sequence which encodes upon expression a feronia gene product operably linked to a promoter to direct or to inhibit expression of the protein. These constructs are then introduced into plant cells using standard molecular biology techniques. The invention can also be used for hybrid plant or seed production, once transgenic inbred parental lines have been established.

In another aspect the invention involves the inhibition of the regulatory gene product in plants through introduction of a construct designed to inhibit the same gene product. The design and introduction of such constructs based upon known DNA sequences is known in the art and includes such technologies as antisense RNA or DNA, co-suppression or any other such mechanism. Several of these mechanisms are described and disclosed in U.S. Pat. No. 5,686,649 to Chua et. al, which is hereby expressly incorporated herein by reference.

The methods of the invention described herein may be applicable to any species of plant.

Production of a genetically modified plant tissue either expressing or inhibiting expression of a structural gene combines the teachings of the present disclosure with a variety of techniques and expedients known in the art. In most instances, alternate expedients exist for each stage of the overall process. The choice of expedients depends on the variables such as the plasmid vector system chosen for the cloning and introduction of the recombinant DNA molecule, the plant species to be modified, the particular structural gene, promoter elements and upstream elements used. Persons skilled in the art are able to select and use appropriate alternatives to achieve functionality. Culture conditions for expressing desired structural genes and cultured cells are known in the art. Also as known in the art, a number of both monocotyledonous and dicotyledonous plant species are transformable and regenerable such that whole plants containing and expressing desired genes under regulatory control of the promoter molecules according to the invention may be obtained. As is known to those of skill in the art, expression in transformed plants may be tissue specific and/or specific to certain developmental stages. Truncated promoter selection and structural gene selection are other parameters which may be optimized to achieve desired plant expression or inhibition as is known to those of skill in the art and taught herein.

The following is a non-limiting general overview of Molecular biology techniques which may be used in performing the methods of the invention.

### Promoters

The constructs, promoters or control systems used in the methods of the invention may include a tissue specific promoter, an inducible promoter or a constitutive promoter.

A large number of suitable promoter systems are available. For example one constitutive promoter useful for the invention is the cauliflower mosaic virus (CaMV) 35S. It has been shown to be highly active in many plant organs and during many stages of development when integrated into the genome of transgenic plants and has been shown to confer expression in protoplasts of both dicots and monocots.

Organ-specific promoters are also well known. For example, the E8 promoter is only transcriptionally activated during tomato fruit ripening, and can be used to target gene expression in ripening tomato fruit (Deikman and Fischer, EMBO J. (1988) 7:3315; Giovannoni et al., The Plant Cell (1989) 1:53). The activity of the E8 promoter is not limited to tomato fruit, but is thought to be compatible with any system wherein ethylene activates biological processes. Similarly the Lipoxegenase (“the LOX gene”) is a fruit specific promoter.

Other fruit specific promoters are the 1.45 promoter fragment disclosed in Bird, et al., Plant Mol. Bio., pp 651-663(1988) and the polygalacturonase promoter from tomato disclosed in U.S. Pat. No. 5,413,937 to Bridges et al. Leaf specific promoters include as the AS-1 promoter disclosed in U.S. Pat. No. 5,256,558 to Coruzzi and the RBCS-3A promoter isolated from pea the RBCS-3A gene disclosed in U.S. Pat. No. 5,023,179 to Lam et al.

And finally root specific promoters include the Cam 35 S promoter disclosed in U.S. Pat. No. 391,725 to Coruzzi et al; the RB7 promoter disclosed in U.S. Pat. No. 5,459,252 to Conking et al and the promoter isolated from Brassica napus disclosed in U.S. Pat. No. 5,401,836 to Bazczynski et al. which give root specific expression.

Other examples of promoters include maternal tissue promoters such as seed coat, pericarp and ovule. Promoters highly expressed early in endosperm development are most effective in this application. Of particular interest is the promoter from the a′ subunit of the soybean β-conglycinin gene [Walling et al., Proc. Natl. Acad. Sci. USA 83:2123-2127 (1986)] which is expressed early in seed development in the endosperm and the embryo.

Further seed specific promoters include the Napin promoter described in U.S. Pat. No. 5,110,728 to Calgene, which describes and discloses the use of the napin promoter in directing the expression to seed tissue of an acyl carrier protein to enhance seed oil production; the DC3 promoter from carrots which is early to mid embryo specific and is disclosed at Plant Physiology, Oct. 1992 100(2) p. 576-581, “Hormonal and Environmental Regulation of the Carrot Lea-class Gene Dc 3, and Plant Mol. Biol., April 1992, 18(6) p. 1049-1063, “Transcriptional Regulation of a Seed Specific Carrot Gene, DC 8”: the phaseolin promoter described in U.S. Pat. No. 5,504,200 to Mycogen which discloses the gene sequence and regulatory regions for phaseolin, a protein isolated from P. vulgaris which is expressed only while the seed is developing within the pod, and only in tissues involved in seed generation.

Other organ-specific promoters appropriate for a desired target organ can be isolated using known procedures. These control sequences are generally associated with genes uniquely expressed in the desired organ. In a typical higher plant, each organ has thousands of mRNAs that are absent from other organ systems (reviewed in Goldberg, Phil, Trans. R. Soc. London (1986) B314-343. mRNAs are first isolated to obtain suitable probes for retrieval of the appropriate genomic sequence which retains the presence of the natively associated control sequences. An example of the use of techniques to obtain the cDNA associated with mRNA specific to avocado fruit is found in Christoffersen et al., Plant Molecular Biology (1984) 3:385. Briefly, mRNA was isolated from ripening avocado fruit and used to make a cDNA library. Clones in the library were identified that hybridized with labeled RNA isolated from ripening avocado fruit, but that did not hybridize with labeled RNAs isolated from unripe avocado fruit. Many of these clones represent mRNAs encoded by genes that are transcriptionally activated at the onset of avocado fruit ripening.

Another very important method that can be used to identify cell type specific promoters that allow even to identification of genes expressed in a single cell is enhancer detection (O'Kane, C., and Gehring, W. J. (1987), “Detection in situ of genomic regulatory elements in Drosophila”, Proc. Natl. Acad. Sci. USA, 84, 9123-9127). This method was first developed in Drosophila and rapidly adapted to mice and plants (Wilson, C., Pearson, R. K., Bellen, H. J., O'Kane, C. J., Grossniklaus, U., and Gehring, W. J. (1989), “P-element-mediated enhancer detection: an efficient method for isolating and characterizing developmentally regulated genes in Drosophila”, Genes & Dev., 3, 1301-1313; Skarnes, W. C. (1990), “Entrapment vectors: a new tool for mammalian genetics”, Biotechnology, 8, 827-831; Topping, J. F., Wei, W., and Lindsey, K. (1991), “Functional tagging of regulatory elements in the plant genome”, Development, 112, 1009-1019; Sundaresan, V., Springer, P. S., Volpe, T., Haward, S., Jones, J. D. G., Dean, C., Ma, H., and Martienssen, R. A., (1995), “Patterns of gene action in plant development revealed by enhancer trap and gene trap transposable elements”, Genes & Dev., 9, 1797-1810).

The promoter used in the method of the invention may be an inducible promoter. An inducible promoter is a promoter that is capable of directly or indirectly activating transcription of a DNA sequence in response to an inducer. In the absence of an inducer, the DNA sequence will not be transcribed. Typically, the protein factor that binds specifically to an inducible promoter to activate transcription is present in an inactive form which is then directly or indirectly converted to the active form by the inducer. The inducer may be a chemical agent such as a protein, metabolite (sugar, alcohol etc.), a growth regulator, herbicide, or a phenolic compound or a physiological stress imposed directly by heat, salt, toxic elements etc. or indirectly through the action of a pathogen or disease agent such as a virus. A plant cell containing an inducible promoter may be exposed to an inducer by externally applying the inducer to the cell such as by spraying, watering, heating, or similar methods. Examples of inducible promoters include the inducible 70 kd heat shock promoter of D. melanogaster (Freeling, M., Bennet, D. C., Maize ADN 1, Ann. Rev. of Genetics, 19:297-323) and the alcohol dehydrogenase promoter which is induced by ethanol (Nagao, R. T., et al., Miflin, B. J., Ed. Oxford Surveys of Plant Molecular and Cell Biology, Vol. 3, p. 384-438, Oxford University Press, Oxford 1986) or the Lex A promoter which is triggered with chemical treatment and is available through Ligand pharmaceuticals. The inducible promoter may be in an induced state throughout seed formation or at least for a period which corresponds to the transcription of the DNA sequence of the recombinant DNA molecule(s).

Another example of an inducible promoter is the chemically inducible gene promoter sequence isolated from a 27 kd subunit of the maize glutathione-S-transferase (GST II) gene. Two of the inducers for this promoter are N,N-diallyl-2,2-dichloroacetamide (common name: dichloramid) or benzyl-═2-chloro-4-(trifluoromethyl)-5-thiazolecarboxylate (common name: flurazole). In addition, a number of other potential inducers may be used with this promoter as described in published PCT Application No. PCT/GB90/00110 by ICI.

Another example of an inducible promoter is the light inducible chlorophyll a/b binding protein (CAB) promoter, also described in published PCT Application No. PCT/GB90/00110 by ICI.

Inducible promoters have also been described in published Application No. EP89/103888.7 by Ciba-Geigy. In this application, a number of inducible promoters are identified, including the PR protein genes, especially the tobacco PR protein genes, such as PR-1a, PR-1b, PR-1c, PR-1, PR-A, PR-S, the cucumber chitinase gene, and the acidic and basic tobacco beta-1,3-glucanase genes. There are numerous potential inducers for these promoters, as described in Application No. EP89/103888.7.

The preferred promoters may be used in conjunction with naturally occurring flanking coding or transcribed sequences of the feronia regulatory genes or with any other coding or transcribed sequence that is critical to pollin tube formation and/or fertilization.

It may also be desirable to include some intron sequences in the promoter constructs since the inclusion of intron sequences in the coding region may result in enhanced expression and specificity. Thus, it may be advantageous to join the DNA sequences to be expressed to a promoter sequence that contains the first intron and exon sequences of a polypeptide which is unique to cells/tissues of a plant critical to female gametophyte development and/or function.

Additionally, regions of one promoter may be joined to regions from a different promoter in order to obtain the desired promoter activity resulting in a chimeric promoter. Synthetic promoters which regulate gene expression may also be used.

The expression system may be further optimized by employing supplemental elements such as transcription terminators and/or enhancer elements.

### Other Regulatory Elements

In addition to a promoter sequence, an expression cassette or construct should also contain a transcription termination region downstream of the structural gene to provide for efficient termination. The termination region or polyadenylation signal may be obtained from the same gene as the promoter sequence or may be obtained from different genes. Polyadenylation sequences include, but are not limited to the Agrobacterium octopine synthase signal (Gielen et al., EMBO J. (1984) 3:835-846) or the nopaline synthase signal (Depicker et al., Mol. and Appl. Genet. (1982) 1:561-573).

### Marker Genes

Recombinant DNA molecules containing any of the DNA sequences and promoters described herein may additionally contain selection marker genes which encode a selection gene product which confer on a plant cell resistance to a chemical agent or physiological stress, or confers a distinguishable phenotypic characteristic to the cells such that plant cells transformed with the recombinant DNA molecule may be easily selected using a selective agent. One such selection marker gene is neomycin phosphotransferase (NPT II) which confers resistance to kanamycin and the antibiotic G-418. Cells transformed with this selection marker gene may be selected for by assaying for the presence in vitro of phosphorylation of kanamycin using techniques described in the literature or by testing for the presence of the mRNA coding for the NPT II gene by Northern blot analysis in RNA from the tissue of the transformed plant. Polymerase chain reactions are also used to identify the presence of a transgene or expression using reverse transcriptase PCR amplification to monitor expression and PCR on genomic DNA. Other commonly used selection markers include the ampicillin resistance gene, the tetracycline resistance and the hygromycin resistance gene. Transformed plant cells thus selected can be induced to differentiate into plant structures which will eventually yield whole plants. It is to be understood that a selection marker gene may also be native to a plant.

### Transformation

A recombinant DNA molecule whether designed to inhibit expression or to provide for expression containing any of the DNA sequences and/or promoters described herein may be integrated into the genome of a plant by first introducing a recombinant DNA molecule into a plant cell by any one of a variety of known methods. Preferably the recombinant DNA molecule(s) are inserted into a suitable vector and the vector is used to introduce the recombinant DNA molecule into a plant cell.

The use of Cauliflower Mosaic Virus (CaMV) (Howell, S. H., et al, 1980, Science, 208:1265) and gemini viruses (Goodman, R. M., 1981, J. Gen Virol. 54:9) as vectors has been suggested but by far the greatest reported successes have been with Agrobacteria sp. (Horsch, R. B., et al, 1985, Science 227:1229-1231).

Methods for the use of Agrobacterium based transformation systems have now been described for many different species. Generally strains of bacteria are used that harbor Modified versions of the naturally occurring Ti plasmid such that DNA is transferred to the host plant without the subsequent formation of tumors. These methods involve the insertion within the borders of the Ti plasmid the DNA to be inserted into the plant genome linked to a selection marker gene to facilitate selection of transformed cells. Bacteria and plant tissues are cultured together to allow transfer of foreign DNA into plant cells then transformed plants are regenerated on selection media. Any number of different organs and tissues can serve as targets from Agrobacterium mediated transformation as described specifically for members of the Brassicaceae. These include thin cell layers (Charest, P. J., et al, 1988, Theor. Appl. Genet. 75:438-444), hypocotyls (DeBlock, M., et al, 1989, Plant Physiol. 91:694-701), leaf discs (Feldman, K. A., and Marks, M. D., 1986, Plant Sci. 47:63-69), stems (Fry J., et al, 1987, Plant Cell Repts. 6:321-325), cotyledons (Moloney M. M., et al, 1989, Plant Cell Repts. 8:238-242) and embryoids (Neuhaus, G., et al, 1987, Theor. Appl. Genet. 75:30-36). It is understood, however, that it may be desirable in some crops to choose a different tissue or method of transformation.

Other methods that have been employed for introducing recombinant molecules into plant cells involve mechanical means such as direct DNA uptake, liposomes, electroporation (Guerche, P. et al, 1987, Plant Science 52:111-116) and micro-injection (Neuhaus, G., et al, 1987, Theor. Appl. Genet. 75:30-36). The possibility of using microprojectiles and a gun or other device to force small metal particles coated with DNA into cells has also received considerable attention (Klein, T. M. et al., 1987, Nature 327:70-73).

It is often desirable to have the DNA sequence in homozygous state which may require more than one transformation event to create a parental line, requiring transformation with a first and second recombinant DNA molecule both of which encode the same gene product. It is further contemplated in some of the embodiments of the process of the invention that a plant cell be transformed with a recombinant DNA molecule containing at least two DNA sequences or be transformed with more than one recombinant DNA molecule. The DNA sequences or recombinant DNA molecules in such embodiments may be physically linked, by being in the same vector, or physically separate on different vectors. A cell may be simultaneously transformed with more than one vector provided that each vector has a unique selection marker gene. Alternatively, a cell may be transformed with more than one vector sequentially allowing an intermediate regeneration step after transformation with the first vector. Further, it may be possible to perform a sexual cross between individual plants or plant lines containing different DNA sequences or recombinant DNA molecules preferably the DNA sequences or the recombinant molecules are linked or located on the same chromosome, and then selecting from the progeny of the cross, plants containing both DNA sequences or recombinant DNA molecules.

Expression of recombinant DNA molecules containing the DNA sequences and promoters described herein in transformed plant cells may be monitored using Northern blot techniques and/or Southern blot techniques known to those of skill in the art.

A large number of plants have been shown capable of regeneration from transformed individual cells to obtain transgenic whole plants. For example, regeneration has been shown for dicots as follows: apple, Malus pumila (James et al., Plant Cell Reports (1989) 7:658); blackberry, Rubus, Blackberry/raspberry hybrid, Rubus, red raspberry, Rubus (Graham et al., Plant Cell Tissue and Organ Culture (1990) 20:35); carrot, Daucus carota (Thomas et al., Plant Cell Reports (1989) 8:354; Wurtele and Bulka, Plant Science (1989) 61:253); cauliflower, Brassica oleracea (Srivastava et al., Plant Cell Reports (1988) 7:504); celery, Apium graveolens (Catlin et al., Plant Cell Reports (1988) 7:100); cucumber, Cucumis sativus (Trulson et al., Theor. Appl. Genet. (1986) 73:11); eggplant, Solanum melonoena (Guri and Sink, J. Plant Physiol. (1988) 133:52) lettuce, Lactuca sativa (Michelmore et al., Plant Cell Reports (1987) 6:439); potato, Solanum tuberosum (Sheerman and Bevan, Plant Cell Reports (1988) 7:13); rape, Brassica napus (Radke et al., Theor. Appl. Genet. (1988) 75:685; Moloney et al., Plant Cell Reports (1989) 8:238); soybean (wild), Glycine canescens (Rech et al., Plant Cell Reports (1989) 8:33); strawberry, Fragaria x ananassa (Nehra et al., Plant Cell Reports (1990) 9:10; tomato, Lycopersicon esculentum (McCormick et al., Plant Cell Reports (1986) 5:81); walnut, Juglans regia (McGranahan et al., Plant Cell Reports (1990) 8:512); melon, Cucumis melo (Fang et al., 86th Annual Meeting of the American Society for Horticultural Science Hort. Science (1989) 24:89); grape, Vitis vinifera (Colby et al., Symposium on Plant Gene Transfer, UCLA Symposia on Molecular and Cellular Biology J. Cell Biochem Suppl (1989) 13D:255; mango, Mangifera indica (Mathews, et al., symposium on Plant Gene Transfer, UCLA Symposia on Molecular and Cellular Biology J. Cell Biochem Suppl (1989) 13D:264);

and for the following monocots: rice, Oryza sativa (Shimamoto et al., Nature (1989) 338:274); rye, Secale cereale (de la Pena et al., Nature (1987) 325:274); maize, (Rhodes et al., Science (1988) 240:204).

In addition regeneration of whole plants from cells (not necessarily transformed) has been observed in apricot, Prunus armeniaca (Pieterse, Plant Cell Tissue and Organ Culture (1989) 19:175); asparagus, Asparagus officinalis (Elmer et al., J. Amer. Soc. Hort. Sci. (1989) 114:1019);

Banana, hybrid Musa (Escalant and Teisson, Plant Cell Reports (1989) 7:665); bean, Phaseolus vulgaris (McClean and Grafton, Plant Science (1989) 60:117); cherry, hybrid Prunus (Ochatt et al., Plant Cell Reports (1988) 7:393); grape, Vitis vinifera (Matsuta and Hirabayashi, Plant Cell Reports, (1989) 7:684; mango, Mangifera indica (DeWald et al., J Amer Soc Hort Sci (1989) 114:712); melon, Cucumis melo (Moreno et al., Plant Sci letters (1985) 34:195); ochra, Abelmoschus esculentus (Roy and Mangat, Plant Science (1989) 60:77; Dirks and van Buggenum, Plant Cell Reports (1989) 7:626); onion, hybrid Allium (Lu et al., Plant Cell Reports (1989) 7:696); orange, Citrus sinensis (Hidaka and Kajikura, Scientia Horiculturae (1988) 34:85); papaya, Carrica papaya (Litz and Conover, Plant Sci Letters (1982) 26:153); peach, Prunus persica and plum, Prunus domestica (Mante et al., Plant Cell Tissue and Organ Culture (989) 19:1); pear, Pyrus communis (Chevreau et al., Plant Cell Reports (1988) 7:688; Ochatt and Power, Plant Cell Reports (1989) 7:587); pineapple, Ananas comosus (DeWald et al., Plant Cell Reports (1988) 7:535);

watermelon, Citrullus vulgaris (Srivastava et al., Plant Cell Reports (1989) 8:300); wheat, Triticum aestivum (Redway et al., Plant Cell Reports (1990) 8:714).

The regenerated plant are transferred to standard soil conditions and cultivated in a conventional manner.

After the expression or inhibition cassette is stably incorporated into regenerated transgenic plants, it can be transferred to other plants by sexual crossing. Any of a number of standard breeding techniques can be used, depending upon the species to be crossed.

It may be useful to generate a number of individual transformed plants with any recombinant construct in order to recover plants free from any position effects. It may also be preferable to select plants that contain more than one copy of the introduced recombinant DNA molecule such that high levels of expression of the recombinant molecule are obtained.

As indicated above, it may be desirable to produce plant lines which are homozygous for a particular gene. In some species this is accomplished rather easily by the use of anther culture or isolated microspore culture. This is especially true for the oil seed crop Brassica napus (Keller and Armstrong, Z. flanzenzucht 80:100-108, 1978). By using these techniques, it is possible to produce a haploid line that carries the inserted gene and then to double the chromosome number either spontaneously or by the use of colchicine. This gives rise to a plant that is homozygous for the inserted gene, which can be easily assayed for if the inserted gene carries with it a suitable selection marker gene for detection of plants carrying that gene. Alternatively, plants may be self-fertilized, leading to the production of a mixture of seed that consists of, in the simplest case, three types, homozygous (25%), heterozygous (50%) and null (25%) for the inserted gene. Although it is relatively easy to score null plants from those that contain the gene, it is possible in practice to score the homozygous from heterozygous plants by southern blot analysis in which careful attention is paid to the loading of exactly equivalent amounts of DNA from the mixed population, and scoring heterozygotes by the intensity of the signal from a probe specific for the inserted gene. It is advisable to verify the results of the southern blot analysis by allowing each independent transformant to self-fertilize, since additional evidence for homozygosity can be obtained by the simple fact that if the plant was homozygous for the inserted gene, all of the subsequent plants from the selfed seed will contain the gene, while if the plant was heterozygous for the gene, the generation grown from the selfed seed will contain null plants. Therefore, with simple selfing one can easily select homozygous plant lines that can also be confirmed by southern blot analysis.

Creation of homozygous parental lines makes possible the production of hybrid plants and seeds which will contain a modified protein component. Transgenic homozygous parental lines are maintained with each parent containing either the first or second recombinant DNA sequence operably linked to a promoter. Also incorporated in this scheme are the advantages of growing a hybrid crop, including the combining of more valuable traits and hybrid vigor.

The following examples serve to better illustrate the invention described herein and are not intended to limit the invention in any way. All references disclosed herein are hereby incorporated in their entirety by reference.

### EXAMPLE 1

The entire pollination/fertilization process includes pollination, pollen germination and growth, pollen tube guidance to the embryo sac, pollen tube disruption usually occurring in one of the synergids (Russell, S. D. 1992), sperm cell release, targetting to the egg and central cell, and finally the fusion of the two pairs of gametes. In the last years, the interest has mainly focused on self-incompatibility-systems (reviewed in Dickinson 2001, Brugiere et al. 2000) and pollen tube guidance (reviewed in Lord 2000, Franklin-Tong 1999). To date, little is known about the complex mechanisms that target the pollen tube to the embryo sac, the interactions between female and the male gametophytes, sperm discharge, the targeting of the sperm cells to egg and central cell, respectively, and gametic and nuclear fusion.

In a screen for mutants affecting the development and function of the female gametophyte (Moore et al. 1997; Moore et al. in preparation), we identified the feronia mutant, which shows normal pollen tube guidance to the micropyle but fails in double fertilization. After reaching the synergid the pollen tube fails to rupture and release the sperm cells. Instead, it keeps growing and winds around the egg apparatus in the micropylar region of the embryo sac suggesting that the female gametophyte plays an active role in sperm discharge. The Ds element disrupts a gene encoding a Protein Phosphatase 2C indicating that the FERONIA protein is a component of a novel signaling pathway between the male and the female gametophyte involved in pollen tube rupture and sperm release.

### EXAMPLE 2

Insertional mutants of Arabidopsis thaliana (ecotype Landsberg erecta) have been generated using the Ac/Ds system described by Sundaresan et al. 1995. In order to isolate gametophytic mutants, a two step screen was devised based on reduced female fertility and reduced female transmission (Moore et al., 1997). The insertion lines were first screened for reduced seed set (30% to 50% reduction) indicating a defect in female fertility. Such a semisterile phenotype can be caused either by (i) inappropriate environmental conditions, (ii) a mutation in a sporophytically acting gene controlling ovule formation that is only partially penetrant, (iii) reciprocal translocations, or (iv) a defect in female gametophyte development or function. Among the listed possibilities only female gametophytic mutants show a non-Mendelian segregation pattern of the Ds-associated kanamycin resistance gene providing a stringent selection criterium (Moore et al. 1997; Howden et al. 1998). If a mutant is specifically affecting the female gametophyte but has no effect on the male, the kanamycin resistance gene will segregate in a ratio of 1:1 rather than the Mendelian ratio of 3:1 (FIG. 1).

### EXAMPLE 3

To identify the female gametophytic mutations within the semisterile lines, we tested (i) the progeny of self-fertilized plants for the segregation distortion of the kanamycin resistance gene, and (ii) for transmission efficiency of the dominant kanamycin resistance marker in reciprocal crosses to the wild type. The transmission efficiency (in percentage) is determined by the ratio of kanamycin resistant to kanamycin sensitive seedlings and determines how much the transmission of the mutant allele is reduced through male and female gametophytes.

The feronia mutant affects predominantly the female gametophyte: the mutant is semisterile with 50% normal seeds, 49% unfertilized ovules, and 1% of seeds that abort early in development (FIG. 2a). We have found that the Ds element in this line is not separable from the feronia phenotype (N>500) suggesting that the feronia mutation is caused by the Ds insertion. Therefore, the kanamycin resistance gene can be used directly to measure segregation and transmission of the feronia mutation. The segregation ratio of kanamycin resistant to kanamycin sensitive seedlings is 1.04:1.00 in the original isolate and 1:18:1.00 in the next generation suggesting a strong heritable gametophytic defect. The transmission efficiency of the kanamycin resistance gene inferonia plants crossed to wild-type pollen is 14.5% of wild-type transmission. If the mutant is used as a pollen parent and crossed to wild-type plants the transmission efficiency of the kanamycin resistance gene is 78.5% (FIG. 2b). Therefore, the feronia mutant shows a predominantly female defect, although male transmission is slightly reduced as well. Despite residual transmission of feronia through both gametophytes, we never obtained homozygous plants. It is, therefore, likely that embryos homozygous for the mutant allele are aborting early during development, i.e. that FERONIA is also essential for embryo development.

### EXAMPLE 4

Ovules of different stages have been isolated from the feronia mutant and compared to wild-type ovules of corresponding stages. If the mutation leads to a defect during female gametophyte development, 50% of the ovules are expected to show aberrations from the normal seven-celled structure, because in a heterozygous mutant only half the ovules are harboring a mutant gametophyte. Inferonia all mature embryo sacs are cytologically indistinguishable form wild-type embryo sacs (FIG. 3a and b). About 24 hrs after pollination all wild-type embryo sacs have initiated the formation of free nuclear endosperm (FIG. 3c). In feronia mutants only half of the ovules have initiated endosperm formation, whereas the other half remains unfertilized (FIG. 3d) despite pollen tube entrance through the micropyle. In these mutant embryo sacs the homo-diploid central cell nucleus (also referred to as secondary endosperm nucleus) has not started to divide, i.e. remained unfertilized, although the pollen tube has entered the embryo sac. In addition, an unusual structure is observed in the micropylar region of the embryo sac [FIG. 3d, see also FIG. 4(a-d)]. This structure is likely formed by the pollen tube after entering the embryo sac. To test this hypothesis we stained mutant and wild-type embryo sacs with aniline blue, a dye labeling callose that is deposited during pollen tube growth. The staining was performed as described in Huilskamp et al. 1995 and results in an intense blue-green fluorescence of the pollen tube. In wild-type ovules the pollen tube terminates in one of the synergids at the micropylar region. In contrast, the pollen tube within a mutant feronia embryo sac keeps growing and winds around the egg apparatus filling the micropylar region. In about 1% of the embryo sacs the pollen tube enters the central cell and proceeds to grow all the way to the chalzal pole (data not shown). These observations suggest that in theferonia mutant the pollen tube does not rupture and the sperm cells are not released. Instead, the pollen tube remains intact and keeps growing within the female gametophyte.

### EXAMPLE 5

To determine whether the feronia phenotype is indeed caused by a defective embryo sac and not the pollen tube, we crossed both wild-type and feronia plants as a female to a line that shows an intense beta-glucuronidase (GUS) expression in the pollen tube. GUS activity can easily be detected using a histochemical staining procedure. In wild-type plants the pollen tube always terminates in the synergid (FIG. 6a). In contrast, the pollen tube fills the micropylar region in about one half of the ovules in the feronia mutant (FIG. 6b). In a plant heterozygous for feronia, we expect that only half of the ovules bear mutant gametophytes consistent with this finding. Ovules fertilized with pollen of the feronia mutant do not show any defect and effect fertilization normally (data not shown). These results suggest that female gametophytes carrying feronia mutant alleles are unable to induce pollen tube rupture and sperm release. Thus, the female gametophyte plays an active role in this process which was previously thought to be of purely mechanical nature: the FERONIA gene product is required for pollen tube rupture and sperm release in the synergid cell.

### EXAMPLE 6

Southern blot analysis with a Ds-specific probe revealed the presence of only one Ds element in the feronia mutant. This element strictly co-segregates with the feronia phenotype (N>500) suggesting that the FERONIA gene is molecularly tagged. The genomic sequences flanking the Ds element have been isolated by TAIL-PCR (Liu et al. 1995; Grossniklaus et al., 1998a) and were confirmed using specific primers. The Ds element maps to chromosome 3 and inserted into a sequence encoding a Protein Phosphatase 2C (PP2C) (SEQ ID NO: 2 or 4 FIG. 8a). PP2Cs are key regulators of their target proteins and are involved in many signaling pathways (FIG. 6). The corresponding chromosomal region of the Arabidopsis thaliana ecotype Columbia has been sequenced by the EU Arabidosis sequencing project. It has been released in public databases with the Accession-No. AL133452, Gene-No. F 26013.110 (SEQ ID NO: 1 FIG. 8b). The FERONIA gene consists of three exons interrupted by two small introns of 71 bp and 164 bp length, respectively. The open reading frame is 1086 bp long, flanked by 5′ and 3′ untranslated regions which have been determined by RACE-PCR (Grossniklaus et al., 1998b) (SEQ ID NO: 3 FIG. 8c). The next open reading frame upstream starts in reverse direction 205 bp from the FERONIA start codon. Therefore, this short intergenic sequence likely contains the promoter regions for the FERONIA PP2C. The Ds insertion created an 8 bp target site duplication, which is separated only by 2 bp from the splice site of the second intron. As the insertion in feronia disrupts a highly conserved region of the PP2C it is likely a null mutation. The molecular nature of FERONIA which identifies it as a protein phosphatase 2C strongly suggests that FERONIA is involved in a signal transduction cascade that induced pollen tube rupture and sperm release.

### EXAMPLE 7

To determine the spatial and temporal expression pattern of FERONIA we performed RT-PCR on a variety of tissues and in situ hybridization (ISH) experiments to developing reproductive tissues. The RT-PCR analysis (not shown) indicated that FERONIA is expressed in a variety of organs throughout plant development. However, using specific probes against the 3′ end of the FERONIA cDNA for ISH (as described in Vielle-Calzada et al. 1999), FERONIA was found to be expressed specifically in the embryo sac of mature ovules and in developing seeds during the reproductive phase of development. In the embryo sac, very strong expression was detected in the synergids (FIG. 5a,b) consistent with the function of FERONIA in this cell type. After fertilization, FERONIA is also expressed in the developing embryo where it is likely to play an essential role as no homozygous plants have been recovered despite a residual transmission through both gametophytes. Taken together, these results show a close correlation of FERONIA expression and phenotype suggesting that FERONIA plays a crucial role in the fertilization process and during embryogenesis.

### EXAMPLE 8

Fertilization in seed plants requires direct interaction between three organisms, the male and female gametophytes and the maternal sporophyte. In lower plants the gametes are motile, but the success of fertilization depends on the availability of water. Higher plants have reduced gametophytes and the gametes are immotile. Therefore, the gametophytes have to be brought into close proximity to achieve fertilization. This is accomplished by the outgrowth of the pollen tube which proceeds through the sporophytic tissue of the female reproductive organs until it reaches the micropyle of the ovule, an opening that allows the pollen tube to access the female gametophyte. After reaching the embryo sac the pollen tube enters the degenerating synergid where it has to discharge the sperm cells. In the feronia mutant the pollen tube fails to rupture and release the sperm cells. Like fungal hyphae or root hairs, the pollen tube is elongating by tip growth (Yang 1998). The pollen tube in a feronia mutant is intact continuing growth and winding around the egg apparatus suggesting that the cessation of growth, pollen tube rupture and sperm release are dependent on FERONIA activity. Only in about 1% of the mutated embryo sacs endosperm formation is initiated (data not shown) suggesting a release of the sperm cells into the central cell. This might either due to an occasional mechanical disruption of the pollen tube or a higher rate of autonomous endosperm formation in theferonia mutant.

Little is known about the interactions between the two gametophytes that control the cessation of pollen tube growth, pollen tube rupture and the sperm cell release. In most angiosperms the pollen tube terminates in one of the synergids. Often, the synergid starts to degenerate even before the pollen tube has reached the embryo sac (Russell 1992, Christensen et al. 1998). This might mechanically facilitate the entry of the pollen tube into the embryo sac or allow the release of attractive substances that guide the pollen tube to the degenerated synergid. Interestingly, the degeneration only occurs after pollination indicating the presence of a long range signal to the female gametophyte induced at pollination.

### EXAMPLE 9

The phenotype of the feronia mutant demonstrates that the pollen tube does not control sperm cell release on its own, but requires a specific signal within the synergid cell. Thus, the Ds element in the feronia mutant disrupts a Protein Phosphatase 2C (PP2C). These enzymes are involved in signal transduction pathways of the plant hormone ABA (reviewed in Rodriguez 1998) and act as negative regulators of mitogen-activated protein kinase (MAPK) pathways in yeast and plants (Meskiene et al. 1998). In plants, MAPK pathways are activated in response to abiotic and biotic stress (Bogre et al. 2000). Both the synergid degeneration and the pollen tube reception are stress situations for the cell and might either be induced or controlled by similar signaling cascades.

The feronia mutant is the first reported component of a novel signaling pathway controlling the direct interaction between the male and the female gametophyte. The feronia phenotype provides new insights in the mechanisms essential for the fertilization process in higher plants. The understanding of the fertilization is of general interest for plant reproduction and its applications. FERONIA opens the possibility to manipulate double fertilization and to direct the release of sperm cells to specific cell types within the female gametophyte. This is of particular interest for the engineering of apomixis technology as the production of viable apomictic seeds often relies on the formation of a sexual endosperm. In many apomicitc species, the appropriate balance of maternal to paternal genomes in the endosperm, which is required for normal development, depends on special adaptations of the fertilization mechanism such as the targeting of both sperms to the central cell (Grossniklaus et al. 1998, 2001, Grossniklaus, 2001).

### REFERENCES

Bogre, L., Meskiene, I., Heberle-Bors, E., and Hirt, H.(2000). Stressing the role of MAP kinases in mitogenic stimulation. Plant Mol Biol 43, 705-18

Brugiere N., Rothstein S. J., and Cui, Y. (2000). Molecular Mechanism of self-recognition in Brassica self-incompatibility. Trend Plant Sci 5, 432-438.

Christensen, C. A., King, E. J., Jordan, J. R., and Drews, G. N. (1997). Megagametogenesis in Arabidopsis wild type and the Gf mutant. Sex Plant Reprod 10,49-64.

Das, A. K., Helps, N. R., Cohen, P. T., and Barford, D. (1996). Crystal structure of the protein serine/threonine phosphatase 2C at 2.0 A resolution. EMBO J. 15, 6798-809.

Dickinson, H. G. (2001). Pollen stigma interactions: so near yet so far. Trends Genet (2000) 16, 373-376.

Drews, G., Lee D., and Christensen C. A. (1998). Genetic Analysis of Female Gametophyte Development and Function. Plant Cell 10, 5-17.

Franklin-Tong, V. E. (1999), Signalling in Pollination. Curr Opin Plant Biol 2, 490-495.

Grossniklaus, U. (2001). From sexuality to apomixis: molecular and genetic approaches. In The Flowering of Apomixis: From Mechanisms to Genetic Engineering, Y. Savidan, J. G. Carman and T. Dresselhaus, eds (Mexico, D. F.: CIMMYT, IRD, European Commission DG VI), pp. 168-211.

Grossniklaus, U., and Schneitz, K. (1998). Genetic and molecular control of ovule development and megagametogenesis. Seminars in Cell & Devl. Biol. 9, 227-238.

Grossniklaus, U., Spillane, C., Page, D. R., and Kohler, C. (2001). Genomic imprinting and seed development: endosperm formation with and without sex. Curr Opin Plant Biol. 4, 21-27.

Grossniklaus, U., Moore, J., and W. Gagliano (1998a). Molecular and genetic approaches to understanding and engineering apomixis: Arabidopsis as a powerful tool. In Advances in Hybrid Rice Technology. Proceedings of the 3rd International Symposium on Hybrid Rice 1996 S. S. Virmani, E. A. Siddiq and K. Muralidharan, eds. (Manila, Philippines: IRRI Press), pp. 187-212.

Grossniklaus, U., Vielle-Calzada, J-P., Hoeppner, M., and Gagliano, W. (1998b). Maternal control of embryogenesis by MEDEA, a Polycomb-group gene in Arabidopsis. Science 280,446-450.

Howden, R., Park, S. K., Moore, J. M., Orne, J., Grossniklaus, U., and Twell, D. (1998). Selection of T-DNA-Tagged Male and Female Gametophytic Mutants by Segregation Distortion in Arabidopsis. Genetics 149, 621-631.

Hülskamp,M., Schneitz, K., and Pruitt, R. E. (1995). Genetic evidence for a long-range activity that directs pollen guidance in Arabidopsis. Plant Cell 7, 57-64.

Klumpp, S., Hanke, C., Donella-Deana, A., Beyer, A., Kellner, R., Pinna, L. A., and Schultz, J. E. (1994). A membrane-bound protein phosphatase type 2C from Paramecium tetraurelia. Purification, characterization, and cloning. J. Biol. Chem. 269, 32774-32780

Koltunow, A. M., Bicknell, R. A., and Chaudhury, A. (1995). Apomixis: molecular strategies for the generation of genetically identical seeds without fertilization. Plant Physiol. 108,1345-1352.

Liu, Y.-G., Mitsukawa, N., Oosumi, T., and Whittier, R. F. (1995). Efficient isolation and mapping of Arabidopsis thaliana T-DNA insert junctions by thermal asymmertric interlaced PCR. Plant J. 8, 457-463.

Lord, E. (2000). Adhesion and cell movement during pollination: Cherchez la femme. Trends Plant Sci 5, 368-373.

Meskiene, I., Bögre, L., Glaser, W., Balog, J., Brandstötter, M., Zwerger, K., Ammerer, G., and Hirt, H. (1998). MP2C, a plant protein phosphatase 2C, functions as a negative regulator of mitogen-activated protein kinase pathways in yeast and plants. PNAS 95, 1938-1943.

Meyer, K., Leube, M. P. and Grill, E. (1994).A protein phosphatase 2C involved in ABA signal transduction in Arabidopsis thaliana Science 264, 1452-1455.

Moore, J. M., Vielle Calzada, J-P., Gagliano, W., and Grossniklaus, U. (1997). Genetic characterization of hadad, a mutant disrupting megagametogenesis in Arabidopsis thaliana. Cold Spring Harbor Symp. Quant. Biol. 62: 35-47.

Pilgrim, D., McGregor, A., Jackle, P., Johnson, T. and Hansen, D. (1995). The C. elegans sex-determining gene fem-2 encodes a putative protein phosphatase. Mol. Biol. Cell 6, 1159-1171.

Rhagavan, V. (1997). Molecular Embryology of flowering plants. Cambridge University Press.

Rodriguez, P. L. (1998). Protein phosphatase 2C (PP2C) function in higher plants. Plant Mol Biol. 38, 919-27

Russell, S. D. (1992). Double fertilization. Int Rev Cytol 104, 357-388.

Russell, S. D. (1994). Fertilization in higher plants. In Stephenson, A. G. and Kao, T.-H. (eds.) Pollen-Pistil Interactions and Pollen Tube Growth. Current Topics in Plant physiology, Vol. 12, American Society of Plant Physiologists

Shiozaki, K. and Russell, P. (1995).Counteractive roles of protein phosphatase 2C (PP2C) and a MAP kinase kinase homolog in the osmoregulation of fission yeast. EMBO J. 14, 492-502.

Sundaresan, V., Springer, P., Volpe, T., Haward, S., Jones, J. D., Dean C., Ma, H., and Martienssen, R.A. (1995). Patterns of gene action in plant development revealed by enhancer trap and gene trap transposable elements. Genes Dev 15, 1797-1810

Vielle-Calzada, J-P., Thomas, J., Spillane, C., Coluccio, A., Hoeppner M. A., and Grossniklaus, U. (1999). Maintenance of genomic imprinting at the Arabidopsis medea locus requires zygotic DDM1 activity. Genes Dev. 13, 2971-2982.

Yang Z. (1998). Signaling tip growth in plants. Curr Opin Plant Biol 1, 525-30.

What is claimed is:

1. An isolated polynucleotide comprising a polynucleotide selected from:
(a) a polynucleotide encoding a FERONIA polypeptide of the sequences shown in SEQ ID NOS 1, 3, or 5;
(b) a polynucleotide which encodes the polypeptide sequence shown in SEQ ID NO: 2, 4, or 6,
(c) a polynucleotide which selectively hybridizes under conditions of high stringency to a polynucleotide of (a) or (b);
(d) a polynucleotide having at least 80% sequence identity with polynucleotides of (a) or (b);
(e) complementary sequences of polynucleotides of (a), (b), or (c); and
(f) a polynucleotide comprising at least 25 contiguous nucleotides from a polynucleotide of (a), (b), (c), or (d).

2. A recombinant expression cassette comprising a polynucleotide selected from the group consisting of:
(a) a polynucleotide encoding a FERONIA polypeptide of the sequences shown in SEQ ID NOS: 1, 3, or 5;
(b) a polynucleotide which encodes the polypeptide sequence shown in SEQ ID NO: 2, 4, or 6,
(c) a polynucleotide which selectively hybridizes under conditions of high stringency to a polynucleotide of (a) or (b);
(d) a polynucleotide having at least 50% sequence identity with polynucleotides of (a) or (b);
(e) complementary sequences of polynucleotides of (a), (b), or (c); and
(f) a polynucleotide comprising at least 25 contiguous nucleotides from a polynucleotide of (a), (b), (c), or (d).

3. A vector comprising a recombinant expression cassette comprising a polynucleotide selected from the group consisting of:
(a) a polynucleotide encoding a FERONIA polypeptide of the sequences shown in SEQ ID NOS: 1, 3, or 5;
(b) a polynucleotide which encodes the polypeptide sequence shown in SEQ ID NOS: 2, 4, or 6,
(c) a polynucleotide which selectively hybridizes under conditions of high stringency to a polynucleotide of (a) or (b);
(d) a polynucleotide having at least 50% sequence identity with polynucleotides of (a) or (b);
(e) complementary sequences of polynucleotides of (a), (b), or (c); and
(f) a polynucleotide comprising at least 25 contiguous nucleotides from a polynucleotide of (a), (b), (c), or (d).

4. A host cell comprising a recombinant expression cassette comprising a polynucleotide selected from the group consisting of:
(a) a polynucleotide encoding a FERONIA polypeptide of the sequences shown in SEQ ID NO: 1, 3, or 5;
(b) a polynucleotide which encodes the polypeptide sequence shown in SEQ ID NO: 2, 4, or 6,
(c) a polynucleotide which selectively hybridizes under conditions of high stringency to a polynucleotide of (a) or (b);
(d) a polynucleotide having at least 50% sequence identity with polynucleotides of (a) or (b);
(e) complementary sequences of polynucleotides of (a), (b), or (c); and
(f) a polynucleotide comprising at least 25 contiguous nucleotides from a polynucleotide of (a), (b), (c), or (d).

5. The host cell of claim 4 wherein the cell is a plant cell.

6. The host cell of claim 5 wherein the cell is selected from the group consisting of maize, Arabidopsis, sorghum, wheat, tomato, soybean, alfalfa, sunflower, canola, cotton, and rice.

7. A transformed plant comprising a polynucleotide selected from the group consisting of:
(a) a polynucleotide encoding a FERONIA polypeptide of the sequences shown in SEQ ID NOS 1, 3, or 5;
(b) a polynucleotide which encodes the polypeptide sequence shown in SEQ ID NO: 2, 4, or 6,
(c) a polynucleotide which selectively hybridizes under conditions of high stringency to a polynucleotide of (a) or (b);
(d) a polynucleotide having at least 50% sequence identity with polynucleotides of (a) or (b);
(e) complementary sequences of polynucleotides of (a), (b), or (c); and
(f) a polynucleotide comprising at least 25 contiguous nucleotides from a polynucleotide of (a), (b), (c), or (d).

8. A plant seed comprising a polynucleotide selected from the group consisting of:
(a) a polynucleotide encoding a FERONIA polypeptide of the sequences shown in SEQ ID NOS: 1, 3, or 5;
(b) a polynucleotide which encodes the polypeptide sequence shown in SEQ ID NOS: 2, 4, or 6,
(c) a polynucleotide which selectively hybridizes under conditions of high stringency to a polynucleotide of (a) or (b);
(d) a polynucleotide having at least 50% sequence identity with polynucleotides of (a) or (b);
(e) complementary sequences of polynucleotides of (a), (b), or (c); and
(f) a polynucleotide comprising at least 25 contiguous nucleotides from a polynucleotide of (a), (b), (c), or (d).

9. A method of altering gametophyte development in a plant comprising:
A) transforming a plant cell with a vector comprising a polynucleotide selected from the group consisting of:
(a) a polynucleotide encoding a FERONIA polypeptide of the sequences shown in SEQ ID NOS: 1, 3, or 5;
(b) a polynucleotide which encodes the polypeptide sequence shown in SEQ ID NOS: 2, 4, or 6,
(c) a polynucleotide which selectively hybridizes under conditions of high stringency to a polynucleotide of (a) or (b);
(d) a polynucleotide having at least 50% sequence identity with polynucleotides of (a) or (b);
(e) complementary sequences of polynucleotides of (a), (b), or (c); and
(f) a polynucleotide comprising at least 25 contiguous nucleotides from a polynucleotide of (a), (b), (c), or (d);
B) growing the plant cell under plant growing conditions; and
C) providing for the expression of said polynucleotide.

10. A method of making FERONIA protein comprising the steps of:
A) expressing a polynucleotide in a recombinantly engineered cell, wherein the polynucleotide is selected from the group consisting of:
(a) a polynucleotide encoding a FERONIA polypeptide of the sequences shown in SEQ ID NOS 1, 3, or 5;
(b) a polynucleotide which encodes the polypeptide sequence shown in SEQ ID NOS 2, 4, or 6,
(c) a polynucleotide which selectively hybridizes under conditions of high stringency to a polynucleotide of (a) or (b);
(d) a polynucleotide having at least 50% sequence identity with polynucleotides of (a) or (b);
(e) complementary sequences of polynucleotides of (a), (b), or (c); and
(f) a polynucleotide comprising at least 25 contiguous nucleotides from a polynucleotide of (a), (b), (c), or (d), operably linked to a promoter sequence; and
B) purifying the enzyme from the plant seed or other plant parts.

11. An isolated polypeptide having FERONIA-like activity and selected from the group consisting of:
(a) a polypeptide comprising the amino acid sequence set forth in SEQ ID NOS 2, 4, or 6;
(b) a polypeptide encoded by a nucleotide sequence comprising the sequence set forth in SEQ ID NO: 1, 3, or 5;
(d) a polypeptide encoded by a nucleotide sequence that has at least 70% sequence identity to the sequence set forth in SEQ ID NO: 1, 3, or 5;
(e) a polypeptide comprising an amino acid sequence having at least 50% sequence identity to the sequence set forth in SEQ ID NO: 2, 4, or 6; and
(f) a polypeptide comprising an amino acid sequence of at least 30 consecutive amino acids of any of (a) through (e).

12. A polynucleotide encoding the polypeptide of claim 20.

13. An isolated regulatory element that is capable of driving transcription in a plant cell, wherein said regulatory element comprises a nucleotide sequence selected from the group consisting of:
a) sequences natively associated with DNA coding for FERONIA from Arabidopsis;
b) a nucleotide sequences comprising the sequence set forth in SEQ ID NO: 3, bases 1 through 53:
c) a sequence that hybridizes to SEQ ID NO: 3 bases 1 through 53 under highly stringent conditions;
d) a sequence having at least 65% sequence identity to SEQ ID NO: 3 bases 1 through 53, wherein the % sequence identity is based on the entire sequence and is determined by GAP version 10 analysis using default parameters.

14. An isolated regulatory element that is capable of driving transcription in a fertilization and/or pollen tube developmental manner, wherein said regulatory element comprises a nucleotide sequence natively associated with DNA coding for FERONIA.

15. The isolated regulatory element of claim 2, wherein said regulatory element is capable of driving transcription in the synergid and endosperm tissues of Arabidopsis.

16. The isolated regulatory element of claim 2 wherein said regulatory element comprises a nucleotide sequence which comprises a TATA box motif.

17. An isolated regulatory element that is capable of driving transcription in a cellular division and/or proliferation-preferred manner, wherein said regulatory element comprises a nucleotide sequence set forth in SEQ ID NOS: 3 bases 1 through 53.

18. A method of generating apomixis in a plant comprising:
introducing to a plant cell a FERONIA PPC encoding sequence, said sequence operable linked to regulatory sequences which provide for expression of said FERONIA gene product in the female gametophyte, ovule or pollen tube so that sperm is released into the central cell;
generating a plant from said plant cell.

19. The method of claim 6 further comprising the step of: inhibiting the native FERONIA gene product of said plant.

20. The method of claim 7 wherein said regulatory sequence is a tissue specific promoter.

21. The methods of claim 9 wherein said tissue specific promoter is a central cell specific promoter.
